(19) United States
(12) Patent Application Publication
(10) Pub. No.: US 2020/0138021 A1
(43) Pub. Date: May 7, 2020

(54) Cleaning and Antimicrobial Composition and Its Uses

(71) Applicant: Spongebath LLC, Astoria, NY (US)

(72) Inventors: Matthew Flannery, Astoria, NY (US); Cordelia Ryan, Astoria, NY (US); Jessica Kate Tyler, New York, NY (US)

(73) Assignee: Spongebath LLC, Astoria, NY (US)

(21) Appl. No.: 16/631,019

(22) PCT Filed: Jul. 12, 2018
(86) PCT No.: PCT/US2018/041864

§ 371 Date: Jan. 14, 2020

Related U.S. Application Data
(60) Provisional application No. 62/532,382, filed on Jul. 14, 2017.

(51) Int. Cl. A01N 37/02; A01N 25/02; A61L 2/18

(52) U.S. Cl. CPC A01N 37/02; A61L 2/18; A01N 25/02

(57) ABSTRACT

The present disclosed subject matter relates to an aqueous cleaning composition consisting essentially of malic acid, citric acid, and water, wherein the ratio of the weight ratio of malic acid to citric acid in the antimicrobial composition is between 1/1 and 50/1, and wherein the concentration of the malic and citric acids mixture in the aqueous cleaning composition is between 1% and 20%. The aqueous cleaning composition provides a minimum 3 log kill of microbes, bacteria, or viruses. The aqueous cleaning composition may optionally further include a pH indicator that provides a visual indication to the user of the effectiveness of the composition. The aqueous cleaning composition may be used to deodorize, clean, sanitize, or disinfect porous or hard surface articles and hard surfaces.

### TECHNICAL FIELD

This invention relates to compositions or formulations of organic acids for use as fast-acting deodorizers, cleaners, sanitizers, or disinfectants on a variety of surfaces and articles. It also relates to methods of using such compositions or formulations.

### BACKGROUND

Germicidal and antiviral compositions comprising carboxylic acids for use as disinfectants are known in the art. However, many of the compositions include a surfactant to enable the composition to have its intended effect. In some instance, the germicidal composition further comprises a metal derivative or polymeric material in order to be effective.

The following references disclose antimicrobial, disinfecting, or antiviral compositions comprising carboxylic acids and their use.
GB 2103089B issued Mar. 12, 1986 to Housain et al. (Kimberly-Clark Corporation) for “Use of Carboxylic Acids as Virucides”.
EP0505763 issued Nov. 6, 1996 to Fresenius AG for “Aqueous disinfectant, its use for inactivation of Hepatitis-B virus, bacterial spores and Legionella pheumophila, and method of disinfection”.
JP-B-3761199 published May 7, 1993 of Fresenius AG for “Aqueous disinfectant, its use for inactivation of Hepatitis-B virus, bacterial spores and Legionella pheumophila, and method of disinfection”.
U.S. Pat. No. 5,665,307 issued Sep. 9, 1997 to Kirschner et al. (Fresenius AG) for “Aqueous Disinfecting Agent”.
U.S. Pat. No. 6,106,771 issued Aug. 22, 2000 to Fitton for “System and Method for Descaling and Disinfecting Dental Waterlines”.
U.S. Pat. No. 6,262,038 issued Jul. 17, 2001 to Pierce et al. for “Germicidal Composition”.
U.S. Pat. No. 7,635,415 issued Dec. 22, 2009 to Lestage et al. for “Regenerable Cleaning Implement for Sanitizing a Surface”,
Wisniewski, Jerzy “Comparison of Virucidal Action of Disinfectants of Aphthous Fever Virus” Med. Wet. Vol. 27(8), 1971, pp 480-482.
Culver, Alicia, et al. “Safer Products and Practices for Disinfecting and Sanitizing Surfaces” May 2, 2014 available from SFEnvironment.org http://www.cleaningforhealthyschools.org/documents/sfe_th_safer_products_and_practices_for_disinfecting.pdf

### SUMMARY OF THE INVENTION

An embodiment of the invention is a concentrated cleaning formulation consisting essentially of malic acid, citric acid, and water, wherein the weight ratio of malic acid to citric acid is between 1/1 and 50/1 wherein total amount of malic acid and citric acid comprises 15% w/v to 60% w/v of the concentrated cleaning formulation, wherein upon diluting the concentrated cleaning formulation with sufficient water, affords an aqueous cleaning composition having 1% w/v to 20% w/v of the malic and citric acids mixture in the resulting aqueous cleaning composition, and wherein said aqueous cleaning composition provides a minimum of 3 log kill of microbes, bacteria, or viruses on or in a porous or hard surface article when treated for at least 30 seconds.

It is preferred that the weight ratio of malic acid to citric acid is between 1/1 and 30/1 and the total amount of malic acid and citric acid comprises 15% w/v to 60% w/v of the concentrated cleaning formulation, wherein upon diluting the concentrated cleaning composition with sufficient water, affords an aqueous cleaning composition having 1.0% w/v to 10.0% w/v of the malic and citric acids mixture in the resulting aqueous cleaning composition. It is preferred that the resulting aqueous cleaning composition comprises 1.0% w/v to 6.0% w/v of the malic and citric acids mixture.

It is more preferred that the concentrated cleaning formulation have a weight ratio of malic acid to citric acid between 1/1 and 10/1, wherein the total amount of malic acid and citric acid comprises 15% w/v to 60% w/v of the concentrated cleaning formulation, and wherein diluting the concentrated cleaning formulation with sufficient water, produces an aqueous cleaning composition having 1.0% w/v to 20.0% w/v of the malic and citric acids mixture in the resulting aqueous cleaning composition. It is even more preferred that the malic and citric acids mixture comprises 1.0% w/v to 10.0% w/v of the resulting aqueous cleaning composition. It is most preferred that the mixture of malic and citric acids comprises 1.0% w/v to 6.0% w/v of the resulting aqueous cleaning composition.

It is an embodiment that the concentrated cleaning formulation optionally further include 0.0001% w/v to 0.1% w/v of a pH indicator. The pH indicator may be an anthocyanin dye, for example a powder, extract, tincture, or concentrate derived from red cabbage. A change in color of the aqueous cleaning composition containing a pH indicator can provide an indication to the user of the need to add additional cleaning composition to maintain effectiveness.

The concentrated formulation may be used to prepare an aqueous cleaning composition for use on hard surfaces as well as porous or hard surface articles, for example, a sponge, toilet brush, mop or other cleaning device, carpet, fabric, cloth, shoes, padding, mats, desk top, table top, counter top, kitchen sink, cabinet, locker, gym equipment, railing, bathroom or kitchen surface, bathroom or kitchen fixture, bathroom sink, bathtub, wash basin, tile, toilet handle, door knob, railing, toilet, toothbrush, denture, hair brush, makeup applicator, baby bottle, pacifier, toy, utensil, tool, appliance, dish washer, washing machine, brewing tank, or industrial or commercial equipment

An embodiment of the invention is an aqueous cleaning composition consisting essentially of malic acid, citric acid, and water, wherein the weight ratio of malic acid to citric acid is between 1/1 and 50/1, wherein the concentration of malic and citric acids mixture is 1% w/v to 20% w/v of the aqueous cleaning composition, whereby an aqueous cleaning composition is produced which provides a minimum 3 log kill of microbes, bacteria, or viruses on or in a porous or hard surface article when treated for at least 30 seconds.

It is preferred that the aqueous cleaning composition have a weight ratio of malic acid to citric acid that is between 1/1 and 10/1, and have a concentration of the malic and citric acids mixture of 1% w/v to 10% w/v in the aqueous cleaning composition. It is more preferred that the concentration of the malic and citric acids mixture is 1% w/v to 6% w/v of the aqueous cleaning composition.

It is an embodiment of the invention that the aqueous cleaning composition optionally further include 0.0001% w/v to 0.1% w/v of a pH indicator. The pH indicator may be an anthocyanin dye, for example a powder, extract, tincture, or concentrate derived from red cabbage. A change in color of the aqueous cleaning composition containing a pH indicator can provide an indication to the user of the need to add additional cleaning composition to maintain effectiveness.

The aqueous cleaning composition may be used to provide deodorizing, cleaning, sanitization, or disinfection on hard surfaces as well as porous or hard surface articles, for example, a sponge, toilet brush, mop or other cleaning device, carpet, fabric, cloth, shoes, padding, mats, desk top, table top, counter top, kitchen sink, cabinet, locker, gym equipment, railing, bathroom or kitchen surface, bathroom or kitchen fixture, bathroom sink, bathtub, wash basin, tile, toilet handle, door knob, railing, toilet, toothbrush, denture, hair brush, makeup applicator, baby bottle, pacifier, toy, utensil, tool, appliance, dish washer, washing machine, brewing tank, or industrial or commercial equipment.

It is an embodiment of the invention to provide a method of making an aqueous cleaning composition comprising the steps of
(a) preparing a concentrated cleaning formulation consisting essentially of malic acid, citric acid, and water, wherein the weight ratio of malic acid to citric acid is between 1/1 and 50/1, wherein total amount of malic acid and citric acid comprises 15% w/v to 60% w/v of the concentrated cleaning composition, and
(b) diluting the concentrated cleaning formulation with sufficient water to obtain an aqueous cleaning composition having 1% w/v to 20% w/v of the malic and citric acids mixture in the resulting aqueous cleaning composition,
   wherein said aqueous cleaning composition provides a minimum 3 log kill of microbes, bacteria, or viruses on or in a porous or hard surface article when treated for at least 30 seconds. It is preferred that the weight ratio of malic and citric acids in the concentrated cleaning formulation is between 1/1 and 30/1. It is more preferred that the weight ratio of malic and citric acids in the concentrated cleaning formulation is between 1/1 and 10/1.

It is an embodiment of the invention that the concentrated cleaning formulation or the resulting cleaning composition prepared by the method optionally further include 0.0001% w/v to 0.1% w/v of a pH indicator. The pH indicator may be an anthocyanin dye, for example a powder, extract, tincture, or concentrate derived from red cabbage.

It is an embodiment of the invention to provide a method of making an aqueous cleaning composition comprising the steps of
(a) providing a powder cleaning formulation consisting essentially of malic acid and citric acid, wherein the weight ratio of malic acid to citric acid is between 1/1 and 50/1, and
(b) dissolving and diluting the powder cleaning formulation in water to obtain an aqueous cleaning composition having 1% w/v to 20% w/v of the malic and citric acids mixture in the resulting aqueous cleaning composition,
   wherein said sanitizing cleaning composition provides a minimum 3 log kill of microbes, bacteria, or viruses on or in a porous or hard surface article when treated for at least 30 seconds. It is preferred that the weight ratio of malic and citric acids in the powder cleaning formulation is between 1/1 and 30/1. It is more preferred that the weight ratio of malic and citric acids in the powder cleaning formulation is between 1/1 and 10/1.

It is an embodiment of the invention that the powder cleaning formulation or the resulting cleaning composition prepared by the method optionally further includes 0.0001% w/v to 0.1% w/v of a pH indicator. The pH indicator may be an anthocyanin dye, for example a powder, extract, tincture, or concentrate derived from red cabbage.

It is an embodiment of the invention to provide a method for deodorizing, cleaning, sanitizing, or disinfecting a porous or hard surface article comprising the steps of,
(a) providing an aqueous cleaning composition consisting essentially of malic acid, citric acid, and water, wherein the weight ratio of malic acid to citric acid is between 1/1 and 50/1, wherein the concentration of malic and citric acids mixture is 1% w/v to 20% w/v of the aqueous cleaning composition,
(b) contacting the porous or hard surface article with sufficient sanitizing cleaning composition to allow for total immersion of the porous or hard surface article for at least 30 seconds,
(c) removing the porous or hard surface article from the aqueous cleaning solution, and
(d) optionally removing excess aqueous cleaning solution from the porous or hard surface article,
   whereby said aqueous cleaning composition provides a minimum 3 log kill of microbes, bacteria, or viruses that may be present in or on said porous or hard surface article. It is preferred that the weight ratio of malic acid to citric acid in the aqueous cleaning composition is between 1/1 and 30/1. It is more preferred that the weight ratio of malic acid to citric acid in the aqueous cleaning composition is between 1/1 and 10/1.

The above method is particularly useful for porous or hard surface articles such as a sponge, toilet brush, mop or other cleaning device, toothbrush, denture, bite plate, hair brush, makeup applicator, baby bottle, pacifier, toy, utensil, tool, fabric, cloth, or padding.

It is an embodiment of the invention that the aqueous cleaning composition prepared optionally further include 0.0001% w/v to 0.1% w/v of a pH indicator. The pH indicator may be an anthocyanin dye, for example a powder, extract, tincture, or concentrate derived from red cabbage

It is an embodiment of the invention to provide a method for deodorizing, cleaning, sanitizing, or disinfecting a porous surface, hard surface, or hard surface article comprising the steps of
(a) providing an aqueous cleaning composition consisting essentially of malic acid, citric acid, and water, wherein the weight ratio of malic acid to citric acid is between 1/1 and 50/1, wherein the concentration of the malic and citric acids mixture is 1% w/v to 20% w/v of the aqueous cleaning, and
(b) treating the porous, surface, hard surface, or hard surface article with sufficient aqueous cleaning composition to allow for complete coverage of the hard surface or hard surface article for at least 30 seconds,
   wherein treating may comprise the steps of spraying, dipping, or submerging with or in the aqueous cleaning composition, and whereby said aqueous cleaning composition provides a minimum 3 log kill of microbes, bacteria, or viruses that may be present on said hard surface or hard surface article. It is preferred that the weight ratio of malic acid to citric acid in the aqueous cleaning composition is 1/1 and 30/1. It is more preferred that the weight ratio of malic acid to citric acid in the aqueous cleaning composition is 1/1 to 10/1.

The above method is particularly useful for treating hard surfaces or hard surface articles such as a desk top, table top, counter top, kitchen sink, cabinet, locker, gym equipment, railing, bathroom or kitchen surface, bathroom or kitchen fixture, bathroom sink, bathtub, wash basin, tile, toilet handle, door knob, railing, toilet, toothbrush, denture, hair brush, makeup applicator, baby bottle, pacifier, toy, utensil, tool, appliance, dish washer, washing machine, brewing tank, or industrial or commercial equipment. The above method is particularly useful for treating porous surfaces such as a sponge, toilet brush, mop or other cleaning device, carpet, fabric, cloth, shoes, padding, or mats.

It is an embodiment of the invention to provide a method for deodorizing, cleaning, sanitizing, or disinfecting a hard surface or hard surface article comprising the steps of
(a) providing an aqueous cleaning composition consisting essentially of malic acid, citric acid, and water, wherein the weight ratio of malic acid to citric acid is between 1/1 and 50/1, wherein the concentration of malic and citric acids mixture is 1% w/v to 20% w/v of the aqueous cleaning composition,
(b) infusing a paper, cloth or synthetic wipe with the aqueous cleaning composition, and
(c) wiping the hard surface or hard surface article with the aqueous cleaning composition treated paper, cloth, or synthetic wipe,
   whereby said aqueous cleaning composition provides a minimum 3 log kill of microbes, bacteria, or viruses that may be present on said hard surface or hard surface article. It is preferred that the weight ratio of malic acid to citric acid in the cleaning composition is between 1/1 and 30/1. It is more preferred that the weight ratio of malic acid to citric acid in the aqueous cleaning composition is 1/1 and 10/1.

The above method is particularly effective for deodorizing, cleaning, sanitizing, or disinfecting hard surface or hard surface articles which are not suitable or not convenient for direct treatment with the an aqueous cleaning composition. Such articles and surfaces include a counter or table top, desk, door knob or handle, railing, locker, gym equipment, industrial or commercial equipment, shopping cart, telephone, computer keyboard surface or other electronic devices, bathroom surface or fixture, kitchen surface, fixture, or appliance, toy, utensil, or other hard surface or hard surface article capable of being wiped. Alternatively, the wipes may be carried for use to clean items when one is in transit.

### DETAILED DESCRIPTION

This invention relates to a multi-purpose aqueous cleaning composition consisting essentially of malic acid, citric acid and water. Optionally, a pH indicator may be included. The acid component of the aqueous cleaning composition is a mixture of malic and citric acids having a ratio of malic acid to citric acid within the range of about 1/1 w/w and 50/1 w/w. Such an aqueous cleaning composition provides favorable cleaning, sanitizing, or disinfecting properties at concentrations of 1% w/v to 20% w/v of the malic and citric acids mixture. Such an aqueous cleaning composition provides at least a 3 log reduction of microbes, bacteria, or viruses at contact times of greater than 30 seconds. It is most cost effective to use the aqueous cleaning composition within those relative concentrations. Desirably, the aqueous cleaning composition will have a ratio of malic acid to citric acid in the range of about 1/1 and 30/1. It is preferred that the ratio be between 1/1 w/w and 20/1 w/w to obtain optimum cleaning, sanitizing, or disinfecting characteristics. It is most preferred to have a weight ratio of malic acid to citric acid within the range of 1/1 w/w to 10/1 w/w.

Even though an aqueous cleaning composition having higher malic acid ratios provides similar sanitizing or disinfecting properties, such a cleaning composition would suffer by having poorer cleaning properties which are provided by the citric acid component. Thus, the ratios recommended for the present invention represent an optimum ratio of malic and citric acid to be used to achieve effective deodorizing, cleaning, sanitizing, or disinfecting properties at lower concentrations. The optimum ratio provides a faster kill time, a lower possibility of damage to surfaces, and a more convenient and safer product for the user. In addition, the recommended mixtures of malic and citric acids are more cost effective to manufacture and provide a superior benefit to the end user.

The aqueous cleaning composition is effective at relatively low concentrations of the malic and citric acids mixture such as between 1.0% w/v and 20% w/v. Preferably, the aqueous cleaning composition has between 1.0% w/v and 10% w/v of the malic and citric acids mixture. Most preferably, the aqueous cleaning composition has between 1.0% w/v and 6.0% w/v of the malic and citric acids mixture.

The aqueous cleaning composition of the present invention typically provides a minimum of 3 log reduction in microbes, bacteria, or viruses at contact times of greater than 30 seconds. However depending on the concentration of the aqueous cleaning composition and depending on the whether the surface to be treated is a hard surface or a porous surface, other contact times may be found to be appropriate in order to achieve a 3 log reduction. It is preferred that a contact time of at least 30 seconds would be used to provide cleaning and sanitization of a hard surface or hard surface article. It is preferred that a contact time of at least 5 minutes would be used to provide cleaning and sanitization of a porous article. It is preferred that a contact time with a hard surface or hard surface article of at least 1 minute would be used when the more stringent effect of disinfection is desired. It is preferred that a contact time with a porous surface of at least 10 minutes would be used to provide disinfection.

Optionally, the aqueous cleaning composition may include a pH indicator, thereby providing the user with a visual indication of the effectiveness of the composition. Such an aqueous cleaning composition would include 0.0001% w/v to 0.1% w/v of the pH indicator. Suitable pH indicators include an anthocyanin dye. Dried red cabbage powder, red cabbage extract or tincture, or red cabbage concentrate are desirable sources of such an anthocyanin dye.

The aqueous cleaning composition consists only of malic acid, citric acid, and water as essential ingredients to the composition. An advantage of the aqueous cleaning composition is that it has favorable deodorizing, cleaning, sanitizing, and disinfecting properties without the addition of other active ingredients such as surfactants, antimicrobial agents, disinfecting agents, and the like. Thus, such additional ingredients are not needed in order to produce aqueous cleaning compositions with desirable properties.

The aqueous cleaning composition may optionally include inert ingredients, such as a pH indicator, which do not materially affect the cleaning, sanitizing, or disinfecting characteristics of the composition. We note that a given pH indicator may have trace amounts of antimicrobial components, such as alcohol, present in extracts or tinctures. However, only 0.0001% w/v to 0.1% w/v of the pH indicator is typically used in the aqueous cleaning composition or cleaning formulation. Thus, the amount of alcohol or other components present in the indicator would be extremely small relative to the aqueous cleaning composition or cleaning formulation and accordingly would be deemed inert for the purpose of deodorizing, cleaning, sanitizing, or disinfecting.

The aqueous cleaning composition is non-toxic, environmentally safe, and comprises all-natural materials. The aqueous cleaning composition may be used as a bath for immersing articles to be deodorized, cleaned, sanitized, or disinfected. It may be used as a fruit or vegetable wash solution. Alternatively, the aqueous cleaning composition may be used as a spray to deodorize, clean, sanitize, or disinfect surfaces. The aqueous cleaning composition can be impregnated into cloths or paper wipes for use as cleaning, sanitizing, or disinfecting wipes.

An embodiment of the invention is a concentrated cleaning formulation consisting essentially of malic acid, citric acid, and water, wherein the weight ratio of malic acid to citric acid is between 1/1 w/w and 50/1 w/w, wherein the total amount of malic acid and citric acid comprises 15% w/v to 60% w/v of the concentrated cleaning formulation. When such a concentrated cleaning formulation is diluted in water to give a concentration of the malic and citric acids mixture of 1% w/v to 20% w/v, the resulting aqueous cleaning composition provides at least a 3.0 log reduction in microbes, bacteria, or viruses after at least 30 seconds without the addition of surfactants or other antimicrobial disinfecting agents. Optionally, the concentrated cleaning formulation may further include a pH indicator, such as an anthocyanin dye, that provides the user with a general indication of the effectiveness of the aqueous cleaning composition derived from the concentrated cleaning formulation.

Another embodiment of the invention is a powder cleaning formulation consisting essentially of malic acid and citric acid, wherein the ratio of malic acid to citric acid is between 1/1 w/w and 50/1 w/w. When such a powder cleaning formulation is diluted in water to give a concentration of the malic and citric acids mixture of 1% w/v to 20% w/v, the resulting cleaning composition provides at least a 3.0 log reduction in microbes, bacteria, or viruses after at least 30 seconds without the addition of surfactants or other antimicrobial disinfecting agents. Optionally, the powder cleaning formulation may further include a pH indicator, such as an anthocyanin dye, that provides the user with a general indication of the effectiveness of the aqueous cleaning composition derived from the powder cleaning formulation.

It is preferred that the ratio of malic acid to citric acid present in the concentrated cleaning formulation or the powder cleaning, formulation is between 1/1 w/w and 30/1 w/w. It is more preferred that the malic acid to citric acid ratio is between 1/1 w/w and 20/1 w/w. It is most preferred that the malic acid to citric acid ratio present in the concentrated cleaning formulation or the powder cleaning formulation is between 1/1 w/w and 10/1 w/w.

Generally, the concentrated cleaning formulation or the powder cleaning formulation is dissolved and/or diluted in water to the appropriate concentration prior to use. Alternatively, the concentrated cleaning formulation or the powder cleaning formulation may be placed directly in a device, such as a dishwasher or washing machine, and the wash cycle initiated to both dissolve the concentrate or powder as well as deodorize, clean, sanitize, or disinfect the device during a wash cycle.

The water used to dissolve or dilute the concentrated cleaning composition or the powder cleaning formulation should be free of substances that will affect the pH of the resulting, aqueous cleaning composition. Distilled water or deionized water is preferred.

Generally, the pH of the aqueous cleaning composition at the recommended concentrations will have a pH between 2.0 and 2.6. Within that range, a pH indicator, such as an anthocyanin dye such as red cabbage powder, red cabbage extract or tincture, or red cabbage concentrate, which may have been added to the aqueous cleaning composition, will generate a specific color, such as pink in the case of red cabbage powder. As the acids are consumed during usage and/or the solution is diluted with additional water, then the pH of the solution rises. As the pH rises, the color of the solution will change providing a visual indication to the user that additional acid needs to be added to the cleaning composition to restore its superior cleaning, sanitizing, and disinfecting properties.

The aqueous cleaning composition may be used to treat a variety of surfaces or articles to provide a minimum of 3 log reduction in microbes, bacteria, or viruses that may be present thereon. Typically, a minimum contact time of greater than 30 seconds provides that level of control. However, depending on the concentration of the aqueous cleaning composition, and depending on whether the surface is a hard surface or porous surface, longer contact times may be required to achieve a particular level of control.

The aqueous cleaning composition may be used as a spray to deodorize, clean, sanitize, or disinfect hard surfaces such as kitchen sinks, cabinets, and counter tops, bathroom sinks, tubs, and toilets, door knobs, appliances, brewing tanks, industrial or commercial equipment, gym equipment, and other hard non-porous surfaces. The aqueous cleaning composition may be used as a spray to deodorize porous surfaces such as cloth, carpet, shoes, padding, mats and the like. Alternatively, the deodorizing, cleaning, sanitizing, and disinfecting of hard, non-porous surfaces may be accomplished by means of a paper, cloth, or synthetic wipe infused with the aqueous cleaning composition. This latter method may be more effective or convenient to clean other hard surfaces on which a spray is less ideal to use, such as a computer keyboard, door handle, telephone, shopping cart, or the like.

The aqueous cleaning composition may be used in a bath to deodorize, clean, sanitize, or disinfect articles by submersion. For such a use, the article is contacted with the liquid for a period of time, for example 10 minutes, and is removed to achieve deodorization, cleaning, sanitization, or disinfection of the article. This method is applicable to both hard surface articles and porous articles. Typical articles that may be deodorized, cleaned, sanitized, or disinfected by this method would include a sponge, cleaning cloth, toilet brush, mop or other cleaning device, carpet, toothbrush, denture, bite plate, hair brush, makeup applicator, baby bottle, pacifier, toy, utensil, tool, fabric cloth, or padding. Generally, less contact time is needed on hard surface articles than porous articles to achieve a 3.0 log reduction of microbes, bacteria, or viruses.

The aqueous cleaning composition may be used to eliminate odors in or on porous and hard surfaces by killing the odor-causing bacteria and by neutralizing scent. Examples of such odors include but are not limited to body odors, urine odors, mildew odors, pet odors, smoke, and other household odors. The aqueous cleaning composition is effective at eliminating odors on various surfaces or articles such as fabric, carpet, rugs, sneakers, furniture, car seats, tables, lockers, and the like. Typically one would spray the article and allow it to dry before use.

As applied to a sponge, a typical procedure for deodorizing, cleaning, sanitizing, or disinfection would comprise the steps of providing a sufficient volume of a suitable aqueous cleaning composition, as described above, to completely immerse the sponge and leaving the sponge in contact with the cleaning composition for a specified period of time, for example 10 minutes using a 6% aqueous cleaning composition. The excess aqueous cleaning composition is then removed from the sponge via a squeezing or expressing means.

The above exemplary process affords a minimum of 3 log reduction in microbes, bacteria, and viruses that may be present in/on the sponge. It is noted that if the sponge remains in contact with the cleaning composition for a longer period of time (e.g. 30 minutes) the solution can act as a disinfectant and provide an even greater reduction in the number of microbes, bacteria, or viruses. Such longer contact times also can provide a reduction in the amount of certain microbes such as mold or spores that may be present in or on the sponge.

An advantage to the use of these aqueous cleaning compositions is that the malic acid and citric acid present in the aqueous composition is non-toxic and relatively non-corrosive at the preferred concentrations. The aqueous cleaning composition aids in the removal of soap scum, grease, grime, rust stains, mold stains, mildew stains, and other stains from the treated articles or surfaces. The aqueous cleaning composition also is a descaler and is odorless, stable and does not emit hazardous or unpleasant fumes. It is comprised of food-grade ingredients that are generally recognized as safe (GRAS). Furthermore, the treated item may be used after treatment without need for further rinsing or cleaning to remove residual aqueous cleaning composition.

### EXAMPLES

### Example 1. Preparation of LB-Broth (1 L)

Tryptone (10 g), yeast extract (5 g), and sodium chloride (10 g) are dissolved in deionized water (950 mL). The medium is adjusted to pH 7.0 using 1N NaOH and the total volume is brought to 1 liter with additional water. The medium is autoclaved on liquid cycle for 20 minutes at 15 psi. The solution is cooled to 55° C. The resulting LB-broth is stored at room temperature or at +4° C.

### Example 2. Preparation of LB-Plates

Tryptone (10 g), yeast extract (5 g), and sodium chloride (10 g) are dissolved in deionized water (950 mL). The medium is adjusted to pH 7.0 using 1N NaOH and the total volume is brought to 1 liter with additional water. Add powdered agar (15 g). The medium is autoclaved on liquid cycle for 20 minutes at 15 psi. The solution is cooled to 55° C. Pour the medium into petri dishes. Allow to harden, then invert. The resulting LB-plates are stored at +4° C. in the dark.

### Example 3

Effectiveness of Sample Solutions on E. coli at Various Exposure Times

E. coli K12 strain was grown at 37° C. in a shaking incubator to OD₆₀₀ nm of 1.0 (estimated 1×10⁹ cells/mL). The equivalent of 10 mL of OD 1.0 cells was collected by centrifugation and re-suspended as a 10× solution of E. coli in 1 ml sterile water.

The MA 2% w/v+CA 1% w/v sample solution was made by combining 1 g MA and 0.5 g CA in a total volume of 50 mL of store bought drinking water. The pH was measured with a pH meter.

The MA 1%+CA 0.5% sample solution was made by combining 5 mL of the MA 2%+1% CA solution above, with 5 mL of store bought drinking water. The pH was measured with a pH meter.

100 μl of the E. coli 10× solution (estimated 1,000,000,000 cells) was added individually to 10 mL of each sample test solution and a control solution of 10 mL water with no acid. Samples were vortexed to mix after adding E. coli, and a stop watch was started immediately. The incubations occurred at room temperature, 21° C., and aliquots were removed at the time points indicated in the table.

At each time point, 100 μl of the test solution was diluted into 9.9 ml of LB broth to stop the reaction. The sample was mixed by vortexing. 100 μl (estimated 100,000 cells) was plated out onto a LB plate, as “Dilution 1”

1 ml of Dilution 1 was added to 9.0 ml of LB broth followed by mixing by vortexing. 100 μl (estimated 10,000 cells) was plated out onto a LB plate, as “Dilution 2”

1 mL of Dilution 2 was added to 9.0 mL of LB broth, followed by mixing by vortexing.
100 μl (estimated 1000 cells) was plated out onto a LB plate, as “Dilution 3”

### For Control Sample Only:

1 mL of Dilution 3 was added to 9.0 mL of LB broth, followed by mixing by vortexing. 100 μl (estimated 100 cells) was plated out onto a LB plate, as “Dilution 4” in triplicate.

Plates were inverted and grown on LB plates overnight

Colonies were counted on the Dilution 4 control plates, and the result of the three plates was averaged, to determine the actual number of viable cells plated as compared to the estimated number.

This average on Dilution 4 of the control sample was 21 colonies, giving the numbers indicated in the table for viable cells plated.

The number of colonies on the Dilution 1, 2 and 3 plates was counted after treatment with each sample solution, and % kill efficiency was calculated according to the number of cells that survived treatment with each sample solution, relative to # viable cells plated on the control plate. Results were recorded in Table 1, below.

TABLE 1

| | | # Viable Cells Plated | | | |
|---|---|---|---|---|---|
| | Time | 21,000 | 2,100 | 210 | |
| Sample | (minutes) | # Cells Grown | | | % Kill |
| MA 2% + CA | 0.5 | TM | TM | 30 | 85.72 |
| 1% in water | 1 | 100 | 10 | 0 | 99.524 |
| pH = 2.13 | 2 | 2 | 0 | 0 | 99.990 |
| | 5 | 0 | 0 | 0 | >99.9953 |
| | 10 | 0 | 0 | 0 | >99.9953 |
| MA 1% + CA | 2 | 38 | 4 | 1 | 99.81 |
| 0.5% in water | 5 | 0 | 0 | 0 | >99.9953 |
| pH = 2.23 | 10 | 0 | 0 | 0 | >99.9953 |
| | 15 | 0 | 0 | 0 | >99.9953% |

TM = too many colonies to count

### Example 4

Effectiveness of Sample Solutions on E. coli at Various Exposure Times

E. coli K12 strain was grown at 37° C. in a shaking incubator to OD₆₀₀ nm of 1.0 (estimated 1×10⁹ cells/mL). The equivalent of 10 mL of OD 1.0 cells was collected by centrifugation and re-suspended as a 10× solution of E. coli in 1 ml sterile water.

A 5% w/v MA solution and a 5% w/v CA solution were made by adding 2.5 g of each individually to a total volume of 50 mL water.

The MA 1.5%+CA 1.5% sample solution was made by combining 3 mL of the 5% MA solution, 3 mL of the 5% CA solution and 4 mL of water, to a total volume of 10 mL.

The MA 0.75%+CA 0.75% sample solution was made by combining 1.5 mL of the 5% MA solution, 1.5 mL of the 5% CA solution and 7 mL of water, to a total volume of 10 mL.

The CA 5% sample solution was made by taking 10 mL of the 5% CA solution.

The pH's were measured with a pH meter.

100 μl of the E. coli 10× solution (estimated 1,000,000,000 cells) was added individually to 10 mL of each sample test solution and a control solution of 10 mls water with no acid. Samples were vortexed to mix after adding E. coli, and a stop watch was started immediately. The incubations occurred at room temperature, 21° C., and aliquots were removed at the time points indicated in the table.

At each time point, 100 μl of the test solution was diluted into 9.9 mL of LB broth to stop the reaction. The sample was mixed by vortexing. 100 μl (estimated 100,000 cells) was plated out onto a LB plate, as “Dilution 1”

1 ml of Dilution 1 was added to 9.0 mL of LB broth followed by mixing by vortexing. 100 μl (estimated 10,000 cells) was plated out onto a LB plate, as “Dilution 2”

1 ml of Dilution 2 was added to 9.0 mL of LB broth, followed by mixing by vortexing. 100 μl (estimated 1000 cells) was plated out onto a LB plate, as “Dilution 3”

### For Control Sample Only:

1 mL of Dilution 3 was added to 9.0 mL of LB broth, followed by mixing by vortexing. 100 μl (estimated 100 cells) was plated out onto a LB plate, as “Dilution 4” in triplicate.

Plates were inverted and grown on LB plates overnight

Colonies were counted on the Dilution 4 control plates, and the result of the three plates was averaged, to determine the actual number of viable cells plated as compared to the estimated number.

This average on Dilution 4 of the control sample was 93.94 colonies, giving the numbers indicated in the table for viable cells plated.

The number of colonies was counted on the Dilution 1, 2 and 3 plates after treatment with each sample solution, and % kill efficiency was calculated according to the number of cells that survived treatment with each sample solution, relative to # viable cells plated on the control plate. Results were recorded in Table 2 below.

TABLE 2

| | | # Viable Cells Plated | | | |
|---|---|---|---|---|---|
| | Time | 93,940 | 9,394 | 939 | |
| Sample | (minutes) | # Cells Grown | | | % Kill |
| MA 1.5% + CA | 2 | TM | TM | 370 | 60.61316 |
| 1.5% in water | 5 | TM | TM | 37 | 96.06132 |
| pH = 2.2 | 10 | 0 | 0 | 10 | 98.93549 |
| MA 0.75% + CA | 10 | TM | TM | 205 | 78.17756 |
| 0.75% in water | 15 | TM | 245 | 30 | 96.80647 |
| pH = 2.23 | | | | | |
| CA 5% | 5 | TM | TM | 940 | 4.194166 |
| in water | 10 | TM | TM | 250 | 73.38727 |
| pH = 2.0 | 15 | TM | TM | 105 | 88.82265 |
| | 30 | TM | 50 | 0 | 99.46775 |
| | 45 | 0 | 0 | 0 | >99.999 |
| | 60 | 0 | 0 | 0 | >99.999 |

TM = too many colonies to count

### Example 5

Effectiveness of Sample Solutions on B. subtilis at Various Exposure Times

B. subtilis was grown at 37° C. in a shaking incubator to OD₆₀₀ nm of 1.0 (estimated 1×10⁹ cells/mL). The equivalent of 10 mL of OD 1.0 cells was collected by centrifugation and re-suspended as a 10× solution of B. subtilis in 1 mL sterile water.

A 5% MA solution and a 5% CA solution were made by adding 2.5 g of each individually to a total volume of 50 mL water.

The MA 1.5%+CA 1.5% sample solution was made by combining 3 mL of the 5% MA solution, 3 mL of the 5% CA solution and 4 mL of water, to a total volume of 10 mL.

The MA 0.75%+CA 0.75% sample solution was made by combining 1.5 mL of the 5% MA solution, 1.5 mL of the 5% CA solution and 7 mL of water, to a total volume of 10 mL.

The CA 5% sample solution was made by taking 10 mL of the 5% CA solution.

The pH's were measured with a pH meter.

100 μl of the E. coli 10× solution (estimated 1,000,000,000 cells) was added individually to 10 mL of each sample test solution and a control solution of water with no acid. Samples were vortexed to mix after adding E. coli, and a stop watch was started immediately. The incubations occurred at room temperature, 21° C., and aliquots were removed at the time points indicated in the table.

At each time point, 100 μl of the test solution was diluted into 9.9 mL of LB broth to stop the reaction. The sample was mixed by vortexing. 100 μl (estimated 100,000 cells) was plated out onto a LB plate, as “Dilution 1”

1 mL of Dilution 1 was added to 9.0 mL of LB broth followed by mixing by vortexing. 100 μl (estimated 10,000 cells) was plated out onto a LB plate, as “Dilution 2”

1 mL of Dilution 2 was added to 9.0 mL of LB broth, followed by mixing by vortexing. 100 μl (estimated 1000 cells) was plated out onto a LB plate, as “Dilution 3”

### For Control Sample Only:

1 mL of Dilution 3 was added to 9.0 mL of LB broth, followed by mixing by vortexing. 100 μl (estimated 100 cells) was plated out onto a LB plate, as “Dilution 4” in triplicate.

Plates were inverted and grown on LB plates overnight

Colonies were counted on the Dilution 4 control plates, and the result of the three plates was averaged, to determine the actual number of viable cells plated as compared to the estimated number.

This average on Dilution 4 of the control sample was 6.667 colonies, giving the numbers indicated in the table for viable cells plated.

The number of colonies was counted on the Dilution 1, 2 and 3 plates after treatment with each sample solution, and % kill efficiency was calculated according to the number of cells that survived treatment with each sample solution, relative to # viable cells plated on the control plate. Results were recorded in Table 3 below.

TABLE 3

| | | # Viable Cells Plated | | | |
|---|---|---|---|---|---|
| | Time | 6.667 | 666 | 66 | |
| Sample | (minutes) | # Cells Grown | | | % Kill |
| MA 1.5% + CA | 1 | 0 | 0 | 0 | >99.98 |
| 1.5% in water | 2 | 0 | 0 | 0 | >99.98 |
| pH = 2.2 | 5 | 0 | 0 | 0 | >99.98 |
| | 10 | 0 | 0 | 0 | >99.98 |
| MA 0.75% + CA | 10 | 0 | 0 | 0 | >99.98 |
| 0.75% in water | 15 | 0 | 0 | 0 | >99.98 |
| pH = 2.23 | | | | | |
| CA 5% | 5 | 0 | 0 | 0 | >99.98 |
| in water | 10 | 0 | 0 | 0 | >99.98 |
| pH = 2.0 | 15 | 0 | 0 | 0 | >99.98 |
| | 30 | 0 | 0 | 0 | >99.98 |
| | 45 | 0 | 0 | 0 | >99.98 |
| | 60 | 0 | 0 | 0 | >99.98 |

### Example 6

Effectiveness of Sample Solutions on E. coli after 5 Minute Exposure

E. coli K12 strain was grown at 37° C. in a shaking incubator to OD₆₀₀ nm of 1.0 (estimated 1×10⁹ cells/mL). The equivalent of 20 mL of OD 1.0 cells was collected by centrifugation and re-suspended as a 10× solution of E. coli in 2 mL sterile water.

A 5% MA solution and a 5% CA solution were made by adding 2.5 g of each individually to a total volume of 50 mL water.

The various sample solutions were made by combining the relevant volumes of the 5% MA solution, the 5% CA solution and water to 10 mL total volume for each sample.

100 μl of the E. coli 10× solution (estimated 1,000,000,000 cells) was added individually to 10 mL of each sample test solution and a control solution of 10 mL water with no acid. Samples were vortexed to mix after adding E. coli, and a stop watch was started immediately. The incubations occurred at room temperature, 21° C., and aliquots were removed at the time points indicated in the table.

At each time point, 100 μl of the test solution was diluted into 9.9 mL of LB broth to stop the reaction. The sample was mixed by vortexing. 100 μl (estimated 100,000 cells) was plated out onto a LB plate in triplicate, as “Dilution 1”

1 ml of Dilution 1 was added to 9.0 ml of LB broth followed by mixing by vortexing. 100 μl (estimated 10,000 cells) was plated out onto a LB plate in triplicate, as “Dilution 2”

1 ml of Dilution 2 was added to 9.0 ml of LB broth, followed by mixing by vortexing. 100 μl (estimated 1000 cells) was plated out onto a LB plate in triplicate, as “Dilution 3”

1 ml of Dilution 3 was added to 9.0 ml of LB broth, followed by mixing by vortexing. 100 μl (estimated 100 cells) was plated out onto a LB plate in triplicate, as “Dilution 4”

Plates were inverted and grown on LB plates overnight

Colonies were counted on the Dilution 4 control plates, and the result of the three plates was averaged, to determine the actual number of viable cells plated as compared to the estimated number.

This average on Dilution 4 of the control sample was 74 colonies, giving the numbers indicated in the table for viable cells plated.

The number of colonies was counted on the Dilution 1, 2, 3 and 4 plates after treatment with each sample solution, and % kill efficiency was calculated according to the number of cells that survived treatment with each sample solution, relative to # viable cells plated on the control plate. Results were recorded in Table 4 below.

TABLE 4

| | # Viable Cells Plated | | | | |
|---|---|---|---|---|---|
| | 74,000 | 7,400 | 740 | 74 | |
| Sample | # Cells Grown | | | | % Kill |
| 5% CA | TM | TM | TM | 17 | 77% |
| 3% CA | TM | 35 | 2.67 | 0 | 99.65% |
| 2% CA | 12 | 1 | 0 | 0 | 99.99% |
| 5% MA | 0 | 0 | 0 | 0 | >99.999% |
| 3% MA | 0 | 0 | 0 | 0 | >99.999% |
| 2% MA | 0 | 0 | 0 | 0 | >99.999% |
| 4.375% CA/0.625% MA | 65 | 7.67 | 0 | 0 | 99.90% |
| 3.75% CA/1.25% MA | 0 | 0 | 0 | 0 | >99.999% |
| 2.5% CA/2.5% MA | 0 | 0 | 0 | 0 | >99.999% |
| 1.25% CA/3.75% MA | 0 | 0 | 0 | 0 | >99.999% |
| 0.625% CA/4.375% MA | 0 | 0 | 0 | 0 | >99.999% |

TM = too many colonies to count
All numbers shown are averages of triplicate repeats

### Example 7

Modified Non-Food Contact Screening Sanitizer Test on Sponges Using SpongeBath™ Sponge Cleaning System (Escherichia coli 0157:H7)

### Description

Five SpongeBath™ sponge cleaners, five blue scrubber sponges, each individually wrapped in a clear plastic pouch. Citric Acid and DL-Malic Acid were provided by Sigma Aldrich (GBL#470210).

### Purpose

To determine whether or not the SpongeBath™ sponge cleaning system kills ≥99.9% of Escherichia coli 0157:H7 present on sponges within 10-minutes.

### Method

Modified ASTM [E 1153-03]; Standard Test Method for Efficacy of Sanitizers Recommended for inanimate non-food contact surfaces

### Results:

See Tables 5 to 8

### Conclusion

Under the condition of this study, Citric acid and DL-Malic Acid formulation with 100 ppm hard water did achieve a ≥99.9% kill for Escherichia coli 01 57:H7 in the modified Non-Food Contact Sanitizer Screening Test in a 10-minute contact time with 1% organic soil.

Test System: Escherichia coli 0157:H7, ATCC #35150

Test Article: 10 grams of Malic Acid+5 grams Citric Acid+473.2 mL of the AOAC Hard Water

Contact Time: 10 minutes

Carriers: 3M Scotch Brite Scrubber Sponge (4.6 in×2.8 in×0.8 in)

Growth Medium: Nutrient Broth [Anatone Broth] [AOAC 955.11A (a)] for preparation of organisms.

Recovery Medium: Neutralization Broth [D/E Neutralizing Broth supplemented with 1N NaOH]

### Test Conditions

1. Contact Time: 10 minutes
2. Organic Soil: 1% bovine serum in the inoculum
3. Test Temperature: Room Temperature
4. Test Diluent: 100 ppm AOAC Hard Water

### Method

### 7.1 Inoculum Preparation

48 to 54 hours Escherichia coli broth culture at 37±1° C. in 10 mL Nutrient Broth, representing transfers originally derived from at least 3 consecutive 24±2 hour transfers, but not more than 30 total transfers. For this final subculture step, a sufficient number of 25×150 mm tubes containing 10 mL of nutrient broth was inoculated for the test procedure and incubated at 37±1° C. for 48 to 54 hours. Nutrient broth test cultures were vortexed mixed for 3-4 seconds and were left to stand for 10 minutes at room temperature before continuing. The upper portion of each culture was removed, leaving behind any debris or clumps, and transferred to a sterile vessel: The cultures were pooled in the vessel and swirled to mix.

### 7.2 Inoculum Enumeration:

The prepared inoculum tubes were vortexed and then allowed to settle for a minimum of 15 minutes. Ten-fold serial dilutions were made by transferring a 1 mL aliquot of each culture into glass test tubes containing 9 mL of sterile saline. A 1 mL aliquot from each dilution tube was plated into sterile petri dishes in duplicate. The plates were poured with TSA and incubated at 37±1° C. for 48 to 54 hours. Colony-forming units were counted using a Darkfield Quebec Colony Counter.

### 7.3 Pre-Treatment of Test Sponges

New test sponges were removed from the packaging and rinsed for 30 seconds with sterile AOAC Hard Water to remove any resident chemical. The SpongeBath™ sponge cleaner was filled with prepared test product to the designated fill line. The paddles were raised and a test sponge was placed between the paddles. The paddles were squeezed together to fully compress the sponges. Then the fully compressed sponges were lowered into the SpongeBath™ sponge cleaner containing cleaning solution. The paddles were released at the bottom into the locked position. Sponges were allowed to pre-treat for 5-minutes.

### 7.4 Inoculation and Sanitization

After pre-treatment, a test sponge was placed onto a sterile petri dish. 2 mL of the inoculum solution was placed onto the sponge. After inoculation the sponge was incubated at 37±1° C. for 35 to 40-minutes. The SpongeBath™ sponge cleaner was drained out and fresh cleaning solution was added to the unit. After incubation, the test sponge was thoroughly rinsed using fresh AOAC Hard Water for 30-seconds before being placed into the respective SpongeBath™ sponge cleaner. The sponge was soaked within the SpongeBath™ sponge cleaner for the 10-minute contact time and the pH was recorded. The sponge was then removed from the SpongeBath™ sponge cleaner and placed into individual vessels containing 300 mL of sterile neutralizer broth and thoroughly mixed.

### 7.5 Enumeration of Surviving Organisms

Within 30 minutes after the addition of the test sponge into the neutralizer broth, survivors were enumerated by transferring two-30 mL aliquots (or four-15 mL aliquots) and two-3.0 mL aliquots from each jar into sterile petri dishes. The plates were poured with TSA and incubated at 37±1° C. for 48 to 54 hours. Colony-forming units were counted using a Darkfield Quebec Colony Counter.

### 7.6 Controls

7.6.1 Inoculated Control Sponges
Same as above in section 7.3 and 7.4 omitting the sanitization step. Pre-treated test sponge was inoculated with 2 mL of inoculum solution. After inoculation, the sponge already placed onto the petri dish was incubated at 37±1° C. for 35 to 40-minutes. After incubation, the sponge was placed into an individual vessel containing 300 mL of sterile neutralizer broth, thoroughly mixed, and survivors were enumerated by transferring, two-3.0 mL aliquots, two-0.3 mL aliquots and two-30 μL aliquots from each jar into sterile petri dishes. The plates were poured with TSA and incubated at 37±1° C. for 48 to 54 hours. Colony-forming units were counted using a Darkfield Quebec Colony Counter.

7.6.2. Qualitative Positive Controls
The vessel from section 7.6.1 after enumeration was incubated at 37±1° C. for 48 to 54 hours. Following incubation, the jars were observed for bacterial growth (turbidity).

7.6.3. Sterility Control

7.6.3.1. Agar Control
Two sterile Petri dishes were poured with sterile TSA from each lot of media used in the test and were incubated along with the test samples.

7.6.3.2. Neutralizer Broth
Two 1.0 mL aliquots of neutralizer broth were from the same lots used in the test were plated with TSA and incubated along—with the test sample.

7.6.3.3. 100 ppm Hard Water
Two 1.0 mL aliquots of hard water from the same lots used in the test were plated with TSA and incubated along with the test sample.
These controls verified that the media were sterile and aseptic technique during carrier transfer process was used.

TABLE 5
Study Results Log Reduction against Escherichia coli O157:H7

| | Date performed/ | Result | Result | Average | % |
|---|---|---|---|---|---|
| Test Product | contact time | (cfu/carrier) | (log₁₀/carrier) | Log₁₀* | Reduction |
| Malic Acid & | Mar. 22, 2017/ | 1.0 × 10² | 2.00 | 1.85 | 99.996% |
| Citric Acid | 10-minutes | 5 × 10¹ | 1.70 | | |

Legend:
*= average of two carriers;
cfu = colony forming units

TABLE 6
Inoculated Control Carrier Results

| | | Result | Result |
|---|---|---|---|
| | Inoculum | (cfu/ | (log₁₀/ |
| Test Organism | Enumeration | carrier) | carrier) |
| Escherichia coli | 5.9 × 10⁸ cfu/mL | 2.1 × 10⁶ | 6.32 |
| O157:H7 | | | |

Legend:
* = average of two carriers;
cfu = colony forming units

TABLE 7
Qualitative Positive Control Results

| Test Organism | Carrier #1 |
|---|---|
| Escherichia coli O157:H7 | (+) Growth |

Legend:
(+) Growth = Typical growth;
(0) = No growth

TABLE 8
Sterility Check Results

| Media | Lot # | Results |
|---|---|---|
| Tryptic Soy Agar | C-173 | Sterile |
| D/e neutralizer Broth | B-140 | Sterile |
| 100 ppm Hard Water | C-4885 | Sterile |

### Example 8

Modified Non-Food Contact Screening Sanitizer Test on Sponges Using SpongeBath′ Sponge Cleaning System (Staphylococcus aureus)

### Description

Five SpongeBath™ sponge cleaners and six blue scrubber sponges (three each wrapped in a clear plastic pouch). Citric Acid and DL-Malic Acid were provided by Sigma Aldrich.

### Purpose

To determine whether or not the SpongeBath™ sponge cleaning system kills ≥99.9% of Staphylococcus aureus present on sponges within 10-minutes.

### Method

Modified ASTM [E 1153-03]; Standard Test Method for Efficacy of Sanitizers Recommended for inanimate non-food contact surfaces

### Results:

See Tables 9 to 12

### Conclusion

Under the condition of this study, Citric acid and DL-Malic Acid formulation with 100 ppm hard water did achieve a ≥99.9% kill for Staphylococcus aureus in the modified Non-Food Contact Sanitizer Screening Test in a 10-minute contact time with 1% organic soil.

Test System: Staphylococcus aureus ATCC #6538
Test Article: 10 grams of Malic Acid+5 grams Citric Acid+473.2 mL of the AOAC Hard Water
Contact Time: 10 minutes
Carriers: 3M Scotch Brite Scrubber Sponge (4.6 in×2.8 in×0.8 in)
Growth Medium: Nutrient Broth [Anatone Broth] [AOAC 955.11A (a)] for preparation of organisms.
Recovery Medium: Neutralization Broth [D/E Neutralizing Broth supplemented with 1N NaOH]

### Test Conditions

1. Contact Time: 10 minutes
2. Organic Soil: 1% bovine serum in the inoculum
3. Test Temperature: Room Temperature
4. Test Diluent: 100 ppm AOAC Hard Water

### Method

### 8.1 Inoculum Preparation

48 to 54 hours Staphylococcus aureus broth culture at 37±1° C. in 10 mL Nutrient Broth, representing transfers originally derived from at least three consecutive 24±2 hour transfers, but not more than 30 total transfers. For this final subculture step, a sufficient number of 25×150 mm tubes containing 10 mL of nutrient broth was inoculated for the test procedure and incubated at 37±1° C. for 48 to 54 hours. Nutrient broth test cultures were vortexed mixed for 3-4 seconds and were left to stand for 10 minutes at room temperature before continuing. The upper portion of each culture was removed, leaving behind any debris or clumps, and transferred to a sterile vessel: The cultures were pooled in the vessel and swirled to mix.

### 8.2 Inoculum Enumeration:

The prepared inoculum tubes were vortexed and then allowed to settle for a minimum of 15 minutes. Ten-fold serial dilutions were made by transferring a 1 mL aliquot of each culture into glass test tubes containing 9 mL of sterile saline. A 1 mL aliquot from each dilution tube was plated into sterile petri dishes in duplicate. The plates were poured with TSA and incubated at 37±1° C. for 48 to 54 hours. Colony-forming units were counted using a Darkfield Quebec Colony Counter.

### 8.3 Pre-Treatment of Test Sponges

New test sponges were removed from the packaging and rinsed for 30 seconds with sterile AOAC Hard Water to remove any resident chemical. The SpongeBath™ sponge cleaner was filled with prepared test product to the designated fill line. The paddles were raised and a test sponge was placed between the paddles. The paddles were squeezed together to fully compress the sponges. Then the fully compressed sponges were lowered into the SpongeBath™ sponge cleaner containing cleaning solution. The paddles were released at the bottom into the locked position. Sponges were allowed to pre-treat for 5-minutes.

### 8.4 Inoculation and Sanitization

After pre-treatment, a test sponge was placed onto a sterile petri dish. 2 mL of the inoculum solution was placed onto the sponge. After inoculation the sponge was incubated at 37±1° C. for 35 to 40-minutes. The SpongeBath™ sponge cleaner was drained out and fresh cleaning solution was added to the unit. After incubation, the test sponge was thoroughly rinsed using fresh AOAC Hard Water for 30-seconds before being placed into the respective SpongeBath™ sponge cleaner. The sponge was soaked within the SpongeBath™ sponge cleaner for the 10-minute contact time and the pH was recorded. The sponge was then removed from the SpongeBath™ sponge cleaner and placed into individual vessels containing 300 mL of sterile neutralizer broth and thoroughly mixed.

### 8.5 Enumeration of Surviving Organisms

Within 30 minutes after the addition of the test sponge into the neutralizer broth, survivors were enumerated by transferring two-30 mL aliquots (or four-15 mL aliquots) and two-3.0 mL aliquots from each jar into sterile petri dishes. The plates were poured with TSA and incubated at 37±1° C. for 48 to 54 hours. Colony-forming units were counted using a Darkfield Quebec Colony Counter.

### 8.6 Controls

8.6.1 Inoculated Control Sponges
Same as above in section 8.3 and 8.4 omitting the sanitization step. Pre-treated test sponge was inoculated with 2 mL of inoculum solution. After inoculation, the sponge already placed onto the petri dish was incubated at 37±1° C. for 35 to 40-minutes. After incubation, the sponge was placed into an individual vessel containing 300 mL of sterile neutralizer broth, thoroughly mixed, and survivors were enumerated by transferring, two-3.0 mL aliquots, two-0.3 mL aliquots and two-30 μL aliquots from each jar into sterile petri dishes. The plates were poured with TSA and incubated at 37±1° C. for 48 to 54 hours. Colony-forming units were counted using a Darkfield Quebec Colony Counter.

8.6.2. Qualitative Positive Controls
The vessel from section 8.6.1 after enumeration was incubated at 37±1° C. for 48 to 54 hours. Following incubation, the jars were observed for bacterial growth (turbidity).

8.6.3. Sterility Control

8.6.3.1. Agar Control
Two sterile Petri dishes were poured with sterile TSA from each lot of media used in the test and were incubated along with the test samples.

8.6.3.2. Neutralizer Broth
Two 1.0 mL aliquots of neutralizer broth were from the same lots used in the test were plated with TSA and incubated along—with the test sample.

8.6.3.3. 100 ppm Hard Water
Two 1.0 mL aliquots of hard water from the same lots used in the test were plated with TSA and incubated along with the test sample.
These controls verified that the media were sterile and aseptic technique during carrier transfer process was used.

TABLE 9
Study Results Log Reduction against Staphylococcus aureus

| | Date performed/ | Result | Result | Average | % |
|---|---|---|---|---|---|
| Test Product | contact time | (cfu/carrier) | (log₁₀/carrier) | Log₁₀* | Reduction |
| Malic Acid & | Apr. 19, 2017/ | 1.9 × 10² | 2.28 | 2.16 | 99.998% |
| Citric Acid | 10-minutes | 1.1 × 10² | 2.04 | | |

Legend:
*= average of two carriers;
cfu = colony forming units

TABLE 10
Inoculated Control Carrier Results

| | | Result | Result |
|---|---|---|---|
| | Inoculum | (cfu/ | (log₁₀/ |
| Test Organism | Enumeration | carrier) | carrier) |
| Staphylococcus aureus | 1.0 × 10⁸ cfu/mL | 7.0 × 10⁶ | 6.85 |

Legend:
* = average of two carriers;
cfu = colony forming units

TABLE 11
Qualitative Positive Control Results

| Test Organism | Carrier #1 |
|---|---|
| Staphylococcus aureus | (+) Growth |

Legend:
(+) Growth = Typical growth;
(0) = No growth

TABLE 12
Sterility Check Results

| Media | Lot # | Results |
|---|---|---|
| Tryptic Soy Agar | C-173 | Sterile |
| D/e neutralizer Broth | B-140 | Sterile |
| 100 ppm Hard Water | C-4885 | Sterile |

### Example 9

### Modified Non-Food Contact Sanitizer Screening Test on Sponges Using SpongeBath′ Concentrated Cleaning Solution at 6%

### Description

Two 8-ounce bottles containing SpongeBath™ Concentrated Cleaning (pink liquid) Lot # BA7178. Two packs each containing three Scotch Brite Heavy Duty Scrub Sponges 3M [Size 4.5″×2.7″×0.6″] and five sponge bath units in white+blue boxes were received on Mar. 1, 2018. Citric Acid and DL-Malic Acid were provided by Brenntag.

### Purpose

To determine whether or not the SpongeBath™ sponge cleaning system kills ≥99.9% of Staphylococcus aureus present on sponges within 1-minute.

### Method

Modified ASTM [E 1153-03]; Standard Test Method for Efficacy of Sanitizers Recommended for inanimate non-food contact surfaces

### Results:

See Tables 13 to 16

### Conclusion

Under the condition of this study, Concentrated Cleaning Solution at 6% with 100 ppm hard water did achieve a ≥99.9% kill for Staphylococcus aureus ATCC #6538 in the modified Non-Food Contact Sanitizer Screening Test in a 1-minute contact time with 1% organic soil.

Test System: Staphylococcus aureus ATCC #6538
Test Article: SpongeBath™ Concentrated Cleaning Solution 6% [20 grams of Malic Acid+10 grams Citric Acid+473.2 mL of the AOAC Hard Water]
Contact Time: 1 minute
Carriers: Scotch Brite Heavy Duty Scrub Sponge (4.5 in×2.7 in×0.6 in)
Growth Medium: Nutrient Broth [Anatone Broth] [AOAC 955.11A (a)] for preparation of organisms.
Recovery Medium: Neutralization Broth [D/E Neutralizing Broth supplemented with 1N NaOH]

### Test Conditions

1. Contact Time: 1 minute
2. Organic Soil: 1% bovine serum in the inoculum
3. Test Temperature: Room Temperature
4. Test Diluent: 100 ppm AOAC Hard Water

### Method

### 9.1 Inoculum Preparation

48 to 54 hours Staphylococcus aureus broth culture at 37±1° C. in 10 mL Nutrient Broth, representing transfers originally derived from at least three consecutive 24±2 hour transfers in 10 mL Nutrient Broth but not more than 30 total transfers. For this final subculture step, inoculate for the test procedure, a sufficient number of 25×150 mm tubes containing 10 mL of nutrient broth was inoculated for the test procedure and incubated at 37±1° C. for 48 to 54 hours. Nutrient broth test cultures were vortexed mixed for 3-4 seconds and were left to stand for 10 minutes at room temperature before continuing. The upper portion of each culture was removed, leaving behind any debris or clumps, and transferred to a sterile vessel: The cultures were pooled in the vessel and swirled to mix.

### 9.2 Inoculum Enumeration:

The prepared inoculum tubes were vortexed and then allowed to settle for a minimum of 15 minutes. Ten-fold serial dilutions were, made by transferring a 1 mL aliquot of each culture into glass test tubes containing 9 mL of sterile saline. A 1 mL aliquot from each dilution tube was plated into sterile petri dishes. The plates were poured with TSA and incubated at 37±1° C. for 48 to 54 hours. Colony-forming units were counted using a Darkfield Quebec Colony Counter.

### 9.3 Pre-Treatment of Test Sponges

Two new test sponges were removed from packaging and rinsed for 30 seconds with sterile AOAC Hard Water to remove any resident chemical. Add 60 mL of product into the SpongeBath™ unit and fill the unit with tap water to designated fill line on back paddle. The Paddles were raised and test sponges were placed between paddles. The paddles are squeezed together to fully compress the sponges. Then the fully compressed sponges are lowered into SpongeBath™ unit containing cleaning solution. Paddles are released at the bottom into the locked position. Sponges were allowed to pre-treat for 5-minutes.

### 9.4 Inoculation and Sanitization

After pre-treatment, test sponges were placed onto sterile petri dish. 2 mL of the inoculum solution was placed onto the sponge. After inoculation the sponge already placed onto petri dish were incubated at 37±1° C. for 35 to 40-minutes. Empty out the SpongeBath™ base unit and added fresh 60 mL cleaning solution and filled unit with tap water to designated fill line on back paddle. After incubation, test sponge was thoroughly rinsed using fresh AOAC Hard Water for 30-seconds before being placed into their respective SpongeBath™ base unit. Sponges were soaked within the SpongeBath™ base unit for 1-minute contact time and recorded ph. Sponges were then removed from SpongeBath™ base unit and placed into individual vessels containing 300 mL of sterile neutralizer broth and thoroughly mixed.

### 9.5 Enumeration of Surviving Organisms

Within 30 minutes after the addition of the test sponge into the neutralizer broth, survivors were enumerated by transferring 2-30 mL aliquots (or 4-15 mL aliquots) and two-3.0 mL aliquots from each jar into sterile petri dishes. The plates were poured with TSA and incubated at 37±1° C. for 48 to 54 hours. Colony-forming units were counted using a Darkfield Quebec Colony Counter.

### 9.6 Controls

9.6.1 Inoculated Control Sponges
Same as above in sections 9.3 and 9.4 omitting sanitization step. Test sponges were inoculated with 2 mL of inoculum solution. After inoculation, sponges already placed onto petri dish were incubated at 37±1° C. for 35 to 40-minutes. After incubation, sponge was placed into individual vessels containing 300 rnL of sterile neutralizer broth, thoroughly mixed and survivors were enumerated by transferring, two-3.0 mL aliquots, two-0.3 mL aliquots and 2-30 μL from each jar into sterile petri dishes. The plates were poured with TSA and incubated at 37±1° C. for 48 to 54 hours. Colony-forming units were counted using a Darkfield Quebec Colony Counter.

9.6.2. Qualitative Positive Controls
The vessel from section 9.6.1 after enumeration was incubated at 37±1° C. for 48 to 54 hours. Following incubation, the jars were observed for bacterial growth (turbidity).

9.6.3. Sterility Control

9.6.3.1. Agar Control
Two sterile Petri dishes were poured with sterile TSA from each lot of media used in the test and were incubated along with the test samples.

9.6.3.2. Neutralizer Broth
Two 1.0 mL aliquots of neutralizer broth were from the same lots used in the test were plated with TSA and incubated along—with the test sample.

9.6.3.3. 100 ppm Hard Water
Two 1.0 mL aliquots of hard water from the same lots used in the test were plated with TSA and incubated along with the test sample. These controls verified that the media were sterile and aseptic technique during carrier transfer process was used.

### 9.7 Calculations

9.7.1 Number of Viable Organisms/Milliliters in the Neutralizer Broth
Determine the number of viable organisms in the neutralizer broth from the test carriers and control carriers. Add the total number of colony forming units on each of the duplicate countable plates from each test sample and divide by 2 or 4 if plated in quadruplicate to obtain the number of organisms surviving treatment per milliliter of neutralizer broth.

9.7.2. Number of Organisms Surviving/Carrier
Multiply the number of organisms surviving per milliliter of neutralizer broth by plate factor to provide the number of organisms surviving per sponge.

9.7.3. Calculate the Mean Log₁₀ Density for Control Sponge.
An average of at least 7.5×10⁵ organisms must have survived on the inoculated control sponge for the test to be valid.

9.7.4. Calculate the Mean Log₁₀ Density for Test Sponge.

9.7.5. Percent Reduction
Use the following equation to calculate the percent reduction:

$$\% reduction = \frac{( a - b ) \times 100}{a}$$

where:
a=number of organisms surviving on the inoculated control sponge [as determined in 9.6.1] and
b=number of organisms surviving on the inoculated test sponge [as determined in 9.4]

TABLE 13
Study Results Log Reduction against Staphylococcus aureus ATCC # 6538

| | Date performed/ | Result | Result | Average | % |
|---|---|---|---|---|---|
| Test Product | contact time | (cfu/carrier) | (log₁₀/carrier) | Log₁₀** | Reduction |
| Concentrated | Mar. 9, 2018/ | 4.5 × 10¹ | 1.65 | 1.57 | 99.996% |
| Cleaning | 1-minute | 3.0 × 10¹ | 1.48 | | |
| Solution at 6%* | | | | | |

Legend:
*pH of sample = 2.14
**= average of two carriers;
cfu = colony forming units

TABLE 14
Inoculated Control Carrier Results

| | | Result | Result |
|---|---|---|---|
| | Inoculum | (cfu/ | (log₁₀/ |
| Test Organism | Enumeration | carrier)* | carrier) |
| Staphylococcus aureus | 8.4 × 10⁷ cfu/mL | 9.6 × 10⁵ | 5.98 |

Legend:
*= average of two carriers;
cfu = colony forming units

TABLE 15
Qualitative Positive Control Results

| Test Organism | Carrier #1 |
|---|---|
| Staphylococcus aureus | (+) Growth |

Legend:
(+) Growth = Typical growth;
(0) = No growth

TABLE 16
Sterility Check Results

| Media | Lot # | Results |
|---|---|---|
| Tryptic Soy Agar | C-83 | Sterile |
| D/E neutralizer Broth | B-156 | Sterile |
| 100 ppm Hard Water | C-5264 | Sterile |

### Example 10

### Preparation of Aqueous Cleaning Composition by Diluting a Concentrated Cleaning Composition.

A concentrated cleaning composition is prepared by combing the respective amounts of malic acid and citric acid in distilled water and diluted to a volume of 8 oz (236.56 mL) to produce the aqueous cleaning compositions as exemplified in Table 17 below. The last column provides the total acids % w/v present in each of the resulting aqueous cleaning compositions that can be prepared by diluting 1 oz. (29.57 mL) of the respective concentrated cleaning compositions to 500 mL with distilled water.

TABLE 17

| | | | | | Acids % Concentrated | Acids % Aqueous | |
|---|---|---|---|---|---|---|---|
| Malic | Citric | Ratio | Total | | Cleaning Composition | Cleaning Composition | |
| Acid | Acid | M/C | Acid | | (8 oz) | (1 oz diluted to 500 mL) | |
| 30.0 g | 10.0 g | 3.0 | 40.0 | g | 17.0% w/v | 1.0% | w/v |
| 30.0 g | 15.0 g | 2.0 | 45.0 | g | 19.0% w/v | 1.125% | w/v |
| 50.0 g | 25.0 g | 2.0 | 75.0 | g | 32.0% w/v | 1.875% | w/v |
| 80.0 g | 40.0 g | 2.0 | 120.0 | g | 50.7% w/v | 3.0% | w/v |
| 60.0 g | 43.0 g | 1.4 | 103.0 | g | 43.5% w/v | 2.58% | w/v |
| 60.0 g | 50.0 g | 1.2 | 110.0 | g | 46.5% w/v | 2.75% | w/v |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the various embodiments stated herein.

Having generally described this invention, those skilled in the art will appreciate that the present invention contemplates the embodiments of this invention as defined in the following claims, and equivalents thereof. However, those skilled in the art will appreciate that the scope of this invention should be measured by the claims appended hereto, and not merely by the specific embodiments exemplified herein. Those skilled in the art will also appreciate that more sophisticated technological advances will likely appear subsequent to the filing of this document with the Patent Office. To the extent that these later developed improvements embody the operative principles at the heart of the present disclosure, those improvements are likewise considered to come within the ambit of the following claims.

1. A concentrated cleaning formulation consisting essentially of malic acid, citric acid, and water, wherein the weight ratio of malic acid to citric acid is between 1/1 and 50/1 wherein total amount of malic acid and citric acid comprises 15% w/v to 60% w/v of the concentrated cleaning formulation, wherein upon diluting the concentrated cleaning formulation with sufficient water, affords an aqueous cleaning composition having 1% w/v to 20% w/v of malic and citric acids mixture in the resulting aqueous cleaning composition, and wherein said aqueous cleaning composition provides a minimum of 3 log kill of microbes, bacteria, or viruses on or in a hard surface or a porous or hard surface article when treated for at least 30 seconds.

2-3. (canceled)

4. The concentrated cleaning formulation of claim 1, wherein the weight ratio of malic acid to citric acid is between 1/1 and 10/1, wherein the total amount of malic acid and citric acid comprises 15% w/v to 60% w/v of the concentrated cleaning formulation, wherein diluting the concentrated cleaning formulation with sufficient water, produces an aqueous cleaning composition having 1.0% w/v to 20.0% w/v malic and citric acids mixture in the resulting aqueous cleaning composition.

5. (canceled)

6. The concentrated cleaning formulation of claim 1, further including 0.0001% w/v to 0.1% w/v of a pH indicator, wherein a change in color of the resulting aqueous cleaning composition provides an indication to the user of the need to add additional cleaning composition to maintain effectiveness.

7-8. (canceled)

9. The concentrated formulation of claim 1, wherein said hard surface or said porous or hard surface article is a sponge, toilet brush, mop or other cleaning device, carpet, fabric, cloth, shoes, padding, mats, desk top, table top, counter top, kitchen sink, cabinet, locker, gym equipment, railing, bathroom or kitchen surface, bathroom or kitchen fixture, bathroom sink, bathtub, wash basin, tile, toilet handle, door knob, railing, toilet, toothbrush, denture, bite plate, hair brush, makeup applicator, baby bottle, pacifier, toy, utensil, tool, appliance, dish washer, washing machine, brewing tank, or industrial or commercial equipment.

10. An aqueous cleaning composition consisting essentially of malic acid, citric acid, and water, wherein the weight ratio of malic acid to citric acid is between 1/1 and 50/1, wherein the concentration of malic and citric acids mixture is 1% w/v to 20% w/v of the aqueous cleaning composition, whereby an aqueous cleaning composition is produced which provides a minimum 3 log kill of microbes, bacteria, or viruses on or in a hard surface or a porous or hard surface article when treated for at least 30 seconds.

11. The aqueous cleaning composition of claim 10, wherein the weight ratio of malic acid to citric acid is between 1/1 and 10/1, wherein the concentration of malic and citric acids mixture is 1% w/v to 10% w/v of the aqueous cleaning composition.

12-15. (canceled)

16. The aqueous cleaning composition of claim 10, wherein said hard surface or said porous or hard surface article is a sponge, toilet brush, mop or other cleaning device, carpet, fabric, cloth, shoes, padding, mats, desk top, table top, counter top, kitchen sink, cabinet, locker, gym equipment, railing, bathroom or kitchen surface, bathroom or kitchen fixture, bathroom sink, bathtub, wash basin, tile, toilet handle, door knob, railing, toilet, toothbrush, denture, hair brush, makeup applicator, baby bottle, pacifier, toy, utensil, tool, appliance, dish washer, washing machine, brewing tank, or industrial or commercial equipment.

17. A method of making an aqueous cleaning composition comprising the steps of
(a) preparing a concentrated cleaning formulation consisting essentially of malic acid, citric acid, and water, wherein the weight ratio of malic acid to citric acid is between 1/1 and 50/1, wherein total amount of malic acid and citric acid comprises 15% w/v to 60% w/v of the concentrated cleaning composition, and
(b) diluting the concentrated cleaning formulation with sufficient water to obtain an aqueous cleaning composition having 1% w/v to 20% w/v of malic and citric acids mixture in the resulting aqueous cleaning composition,
wherein said aqueous cleaning composition provides a minimum 3 log kill of microbes, bacteria, or viruses on or in a porous or hard surface article when treated for at least 30 seconds.

18. The method of claim 17, wherein the weight ratio of malic and citric acids in the concentrated cleaning formulation is between 1/1 and 30/1.

19. The method of claim 17, wherein the weight ratio of malic and citric acids in the concentrated cleaning formulation is between 1/1 and 10/1.

20-22. (canceled)

23. A method of making an aqueous cleaning composition comprising the steps of
(a) providing a powder cleaning formulation consisting essentially of malic acid and citric acid, wherein the weight ratio of malic acid to citric acid is between 1/1 and 50/1, and
(b) dissolving and diluting the powder cleaning formulation in water to obtain an aqueous cleaning composition having 1% w/v to 20% w/v of malic and citric acids mixture in the resulting aqueous cleaning composition,
wherein said aqueous cleaning composition provides a minimum 3 log kill of microbes, bacteria, or viruses on or in a porous or hard surface article when treated for at least 30 seconds.

24. (canceled)

25. The method of claim 23, wherein the weight ratio of malic and citric acids in the powder cleaning formulation is between 1/1 and 10/1.

26-28. (canceled)

29. A method for deodorizing, cleaning, sanitizing, or disinfecting a porous or hard surface article comprising the steps of
(a) providing an aqueous cleaning composition consisting essentially of malic acid, citric acid, and water, wherein the weight ratio of malic acid to citric acid is between 1/1 and 50/1, wherein the concentration of malic and citric acids mixture is 1% w/v to 20% w/v of the aqueous cleaning composition,
(b) contacting the porous or hard surface article with sufficient sanitizing cleaning composition to allow for total immersion of the porous or hard surface article for at least 30 seconds,
(c) removing the porous or hard surface article from the aqueous cleaning solution, and
(d) optionally removing excess aqueous cleaning solution from the porous or hard surface article,
whereby said aqueous cleaning composition provides a minimum 3 log kill of microbes, bacteria, or viruses that may be present in or on said porous or hard surface article.

30. (canceled)

31. The method of claim 29, wherein the weight ratio of malic acid to citric acid in the aqueous cleaning composition is between 1/1 and 10/1.

32-34. (canceled)

35. The method of claim 29, wherein the porous or hard surface article is a sponge, toilet brush, denture, mop or other cleaning device, carpet, toothbrush, hair brush, makeup applicator, baby bottle, pacifier, toy, utensil, tool, fabric, cloth, or padding.

36. A method for deodorizing, cleaning, sanitizing, or disinfecting a porous surface, hard surface, or hard surface article comprising the steps of
(a) providing an aqueous cleaning composition consisting essentially of malic acid, citric acid, and water, wherein the weight ratio of malic acid to citric acid is between 1/1 and 50/1, wherein the concentration of malic and citric acids mixture is 1% w/v to 20% w/v of the aqueous cleaning composition, and
(b) treating the porous surface, hard surface, or hard surface article with sufficient aqueous cleaning composition to allow for complete coverage of the porous surface, hard surface, or hard surface article for at least 30 seconds,
wherein treating may comprise the steps of spraying, dipping, or submerging with or in the aqueous cleaning composition, and whereby said aqueous cleaning composition provides a minimum 3 log kill of microbes, bacteria, or viruses that may be present on said hard surface or hard surface article.

37. (canceled)

38. The method of claim 36 wherein the weight ratio of malic acid to citric acid in the aqueous cleaning composition is 1/1 to 10/1.

39. The method of claim 36, wherein the hard surface or hard surface article is a desk top, table top, counter top, kitchen sink, cabinet, locker, gym equipment, railing, bathroom or kitchen surface, bathroom or kitchen fixture, bathroom sink, bathtub, wash basin, tile, toilet handle, door knob, railing, toilet, toothbrush, denture, hair brush, makeup applicator, baby bottle, pacifier, toy, utensil, tool, appliance, dish washer, washing machine, brewing tank, or industrial or commercial equipment, and wherein the porous surface is a sponge, toilet brush, mop or other cleaning device, carpet, fabric, cloth, shoes, padding, or mats.

40. (canceled)

41. A method for deodorizing, cleaning, sanitizing, or disinfecting a hard surface or hard surface article comprising the steps of
(a) providing an aqueous cleaning composition consisting essentially of malic acid, citric acid, and water, wherein the weight ratio of malic acid to citric acid is between 1/1 and 50/1, wherein the concentration of malic and citric acids mixture is 1% w/v to 20% w/v of the aqueous cleaning composition,
(b) infusing a paper, cloth or synthetic wipe with the aqueous cleaning composition, and
(c) wiping the hard surface or hard surface article with the aqueous cleaning composition treated paper, cloth, or synthetic wipe, and
whereby said aqueous cleaning composition provides a minimum 3 log kill of microbes, bacteria, or viruses that may be present on said hard surface or hard surface article.

42-43. (canceled)

44. The method of claim 41, wherein the hard surface or hard surface article is a counter or table top, desk, door knob or handle, railing, locker, gym equipment, industrial or commercial equipment, shopping cart, telephone, computer keyboard surface or other electronic devices, bathroom surface or fixture, kitchen surface, fixture, or appliance, toy, utensil, or other hard surface or hard surface article capable of being wiped.
